(19) United States
(12) Patent Application Publication
(10) Pub. No.: US 2014/0250942 A1
(43) Pub. Date: Sep. 11, 2014

(54) STABLE SOLUTIONS OF SACROSIDASE

(71) Applicant: QOL MEDICAL LLC, Vero Beach, FL (US)

(72) Inventors: Dayton T. Reardan, Shorewood, MN (US); Brian L. Miller, Eden Prairie, MN (US)

(73) Assignee: QOL MEDICAL LLC, VERO BEACH, FL (US)

(21) Appl. No.: 13/786,956

(22) Filed: Mar. 6, 2013

(51) Int. Cl. C12N 9/96

(52) U.S. Cl. CPC C12N 9/96
USPC 62/440; 435/188; 428/34.1

(57) ABSTRACT

A solution of sacrosidase in glycerol/water that is enzymatically stable indefinitely when maintained at about −18° C. to −22° C. is disclosed.

### BACKGROUND OF THE INVENTION

Congenital sucrose-isomaltase deficiency (CSID) is a chronic, autosomal recessive, inherited, phenotypically heterogenous disease with very variable enzyme activity. CSID is usually characterized by a complete or almost complete lack of endogenous sucrase activity, a very marked reduction in isomaltase activity, a moderate decrease in maltase activity and normal lactase levels.

Sucrase is naturally produced in the brush border of the small intestine, primarily the distal duodenum and jejunum. Sucrase hydrolyzes the disaccharide surcrose into its component monosaccharides, glucose and fructose. Isolmaltase breaks down disaccharides from starch into simple sugars. Sucraid does not contain isomaltase.

In the absence of endogenous human sucrose, as in CSID, sucrose is not metabolized. Unhydrolyzed sucrose and starch are not absorbed from the intestine and their presence in the intestinal lumen may lead to osmotic retention of water. This may result in loose stools. Unabsorbed sucrose in the colon is fermented by bacterial flora to produce increased amounts of hydrogen, methane and water. As a consequence, excessive gas, bloating, abdominal cramps, nausea and vomiting may occur. Chronic malabsorption of disaccharides may result in malnutrition. Undiagnosed/untreated CSID patients often fail to thrive and fall behind in their expected growth and development curves. Previously, the treatment of CSID has required the continued use of a strict sucrose-free diet.

CSID is currently treated by the oral administration, with meals, of a glycerol-water (1:1 w/w) solution of sacrosidase, which provides an enzyme replacement therapy for CSID. This solution is commercially provided as Sucraid® distributed by QOL Medical LLC.

Each milliliter (mL) of Sucraid® contains 8500 International Units (I.U.) of the enzyme sacrosidase, the active ingredient. The chemical name of this enzyme is β,D-fructofuranoside fructohydrolase. The enzyme is derived from baker's yeast (Saccharomyces cerevisiae).

It has been reported that the primary acid structure of this protein consists of 513 amino acids with an apparent molecular weight of 100,000 g/mole for the glycosolated monomer (range 66,000-116,000 g/mole). Reports also suggest that the protein exists in solution as a monomer, dimer, tetramer, and octomer ranging from 100,000 g/mole to 800,000 g/mole. It has an isoelectric point of 4 (pI=4.093).

Presently, Sucraid® is provided in bottles containing 118 ml of the sacrosidase solution. A typical dose is about 1-2 ml. The solution is bottled aseptically, however, it may become contaminated due to the necessity of frequent administration, so patients are instructed to discard the bottle 4 weeks after opening.

Sacrosidase is also thermally unstable. While patients are advised to store Sucraid® at refrigerator temperatures of about 2-4° C., it has been determined that the shelf life of the formulated drug product is limited to about 24 months (−20.6 IU/ml/month). Although refrigeration would appear to impart substantial stability, the FDA approved stability of the drug substance, sacrosidase, is only 12 months. Combined, the overall shelf life currently approved by FDA is 36 months for the drug substance/drug product combination. This makes logistics warehousing, and inventory planning challenging, short term and difficult to prevent back orders without waste (disposing of “expired” drug). QOL Medical has faced production lead time challenges related to changes in drug substance and drug product manufacturer, sourcing of components, and the time required to develop, build, and approve regulatory options. The company has over the last 5 years twice faced potential drug shortage or back order situations that have only been solved by agreement between the company and the regulators (FDA). These challenges put a strain on reliable supplies, making a method that can extend the shelf life of sacrosidase/Sucraid® formulation desirable.

### SUMMARY OF THE INVENTION

The present invention provides solutions of sacrosidase, e.g., Sucraid®, that are stable for at least about 72 months, preferably for at least about 96 months, e.g., indefinitely, with no detectable loss of enzymatic activity. This remarkable level of stability is provided by maintaining a sacrosidase solution in an aqueous medium of about 0.5-1.25:1, e.g., about 1:1, glycerol:water at a pH of about 4.5 at about −18° C. to about −22° C., preferably at about −20° C., without freezing the solution, or subjecting it to freeze-thaw cycles. For example, reference sacrosidase solutions prepared from Sucraid® by filtering and placing 1.0 ml aliquots in glass bottles and storing them at about −20° C. at strengths of sacrosidase of about 7,500 IU/ml to about 9,500 IU/ml, preferably of about 8,175 IU/ml to 9,000 IU/ml, e.g., about 8500 IU/ml, exhibited a common slope of +1.02 IU/ml/month after storage for 100 months. This indicates no loss of potency and predicted shelf lives of 537 to 713 mos.

Evaluation of two separate lots of reference standard over 13 years led to the discovery that practically infinite stability could be achieved if Sucraid® was stored at about −20° C. Since this temperature is readily achievable in commercial and home freezer units, this discovery should lead to savings and convenience for both the manufacturer, distributor and the ultimate consumer.

This temperature is close to the freezing point of an about 35-55%, e.g., about 45-50%, solution of glycerol in water containing about 8500 IU of sacrosidase/ml, which was determined to be −27° C. to −28° C. However, while it was found that Sucraid®/sacrosidase formulations could be freeze-thawed between about −80° C. and 4° C., loss of activity occurred after 3 freeze-thaw cycles. Therefore, the Sucraid®/sacrosidase formulations should be stored in the liquid state at about −20° C. and not subjected to freezing or to freeze/thaw cycles. Therefore, the present invention provides a system comprising a container as disclosed herein, comprising closure means as disclosed herein, enclosing solution of sacrosidase having an enzymatic activity of about 7500-9500 IU/mL, in about 0.5-1.25:1, e.g., about 1:1, glycerol/water (w/w).

The container can be contained in a refrigeration means, such as a freezer or cooler/dry-ice pack, wherein the solution is maintained at a constant temperature of about −18° C. to about −22° C., e.g., at about −20° C.

Sucraid® contains no preservatives, and is packaged under aseptic conditions. Although the current labeling of Sucraid® indicates that any remaining solution should be discarded 4 weeks after opening due to the potential for bacterial growth, it was unexpected that under ambient conditions, e.g., during 22° C. storage, the formulation could self-sterilize, even after inoculation with a variety of bacteria. Therefore, the present invention also provides a method to maintain a solution of sacrosidase, e.g., Sucraid®, free of bacterial contamination, or to render a sarcosidase solution free of bacterial contamination, by storing the packaged, e.g., bottled solution at about 25° C. for at least about 24 hours to about 3 weeks or more.

It is believed that the present method and system can be used to preserve the activity of a wide variety of enzymes that are unstable temperatures at or above about 0° C., particularly those used in enzyme replacement therapies.

As used herein, the term “about” as used with respect to temperature includes the normal variations of temperature within a conventional refrigeration or freezer, e.g., of about ±4° C., preferably about ±2° C.

The phrase “free of microbial contamination” is to be read in the context of the standard methodologies shown in the Example.

Suitable containers, closure means and refrigeration means are described herein.

### BRIEF DESCRIPTION OF THE INVENTION

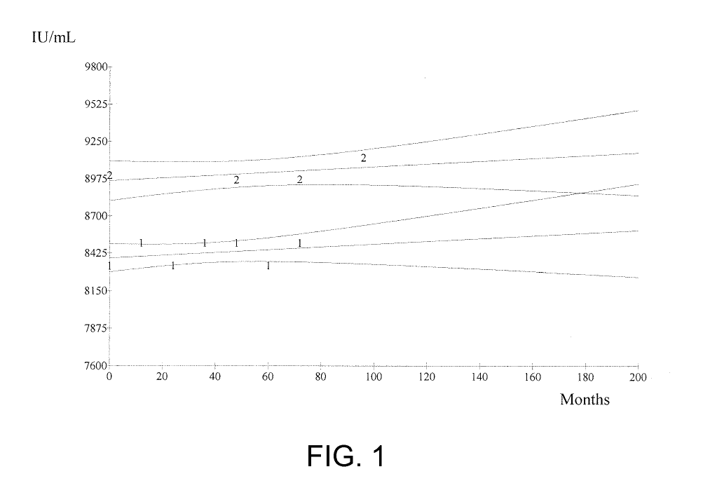

FIG. 1 is a graph depicting the stability of the activity of a sacrosidase solution reference standard (1) and a Sucraid® reference standard (2) held at −20° C., projected to 200 mos.

### DETAILED DESCRIPTION OF THE INVENTION

The present invention provides a protein formulation medium that serves as an effective stabilizing and antimicrobial formulation that is independent of container closure. The present invention provides a protein formulation medium that provides for near infinite retention of protein activity, integrity, and potency as evidenced by preservation of enzymatic activity in a super-cooled liquid state, while retaining the flexibility to return drug substance and drug product to refrigerated temperature distribution channels to accommodate marketing and distribution demands, i.e., to achieve vastly improved logistics. The present invention provides a protein formulation medium that provides for near infinite retention of enzymatic activity in a super-cooled liquid state independent of container/closure system, allowing for flexibility post manufacturing storage of drug substance, filling of drug product containers, and flexibility in labeling.

This invention addresses the problem of producing a liquid protein formulation that has suitable stability to retain enzymatic activity long enough to effectively manage supply chain obstacles to drug substance and drug product manufacture. Previous examples of stabilizing protein formulations for later use without reconstitution have involved freezing the formulation to −80° C. While technically possible, this type of approach is not economically feasible due to the expense of maintaining a −80° C. supply chain, and due to protein inactivating shear forces introduced during freezing and thawing of protein solutions.

An enzyme solution is prepared using sterile filtered water in an aqueous buffer and optionally concentrated to a desired level via ultrafiltration. The resultant solution is combined with an equivalent mass of glycerol, resulting in an approximately 50% glycerol/buffer (w/w) formulation. The formulation is filtered through successive 1.0 micron and 0.45 micron filters, and stored in high density polyethylene drums. The drums are moved to −20° C. cold storage, where the formulation is maintained in a super-cooled liquid state, with the enzyme not suffering any of the damages of freezing and thawing, experiencing no loss of activity that it would otherwise be susceptible to when held at refrigerator temperatures (about 2-8° C.) or held at room temperature (about 20-25° C.) in a formulated state, and not needing reconstitution prior to the next manufacturing step. When the enzyme formulation is needed to prepare drug product, it is allowed to warm to room temperature and then further processed. The further processing can include aseptic filling or additional formulating. Aseptic filling can be performed in glass, LDPE, or some other suitable plastic. In the interim, the protein solution has not degraded due to its near infinite stability in the −20° C. state.

In another iteration, the formulation is stored in low density polyethylene containers or some other suitable plastic containers.

In another iteration, the formulation is stored in glass containers.

In another iteration, drug product prepared using an about 40%-60% glycerol/buffer formulation is stored unlabeled at −20° C. When the drug product is needed for marketing, it is allowed to warm to room temperature, and then labeled. In the interim, the drug product has not degraded due to near infinite stability in the −20° C. state.

### EXAMPLE 1

### Sacrosidase Drug Substance Maintained at 2-8° C.

Sacrosidase drug substance was prepared from saccharomyces cerevisiae yeast and formulated at pH 4.5 in water with approximately 45% (range 40-55%) glycerol.

Stability samples were stored at 4° C. or 2-8° C. (nominal 2-8° C.) in 500 mL HDPE bottles with no colorant. Samples from five lots were tested from 0 to 12 months. Samples from five lots were tested from 0 to 6 months. The samples initially contained between about 8600 IU/ml to 19,500 IU/ml. Stability testing consisted of an enzymatic polarimetry assay that monitors the course of the Sucrose to Glucose+Fructose transformation using the sodium D line at 589 nm.

SLIMSTAT statistical software, by H&A Scientific, Greenville, N.C., was used to model stability results. SLIMSTAT projections indicate common slopes, but different intercepts for each of the drug substance lots.

Based on statistical analysis, a data model 2, common slope but different intercept resulted in a common slope of −17.1 IU/mL/month. This loss of potency limits the shelf life of the drug substance to 12 months even when stored at about 2-8° C., to accommodate the drug product storage needs of the drug product Sucraid®.

The fact that the drug substance, sacrosidase, and drug product, Sucraid®, share the same glycerol/water formulation media means that any degradation phenomena, i.e., loss of activity, is a shared mechanism with shared consequences. Specifically, the FDA approved shelf life for the drug substance was originally only 6 months in order to accommodate an overall stability including drug product of 30 months. This was increased to 12 months for drug substance in 2011 by FDA upon request and provision of data by QOL Medical, while the approved shelf life for the drug product is 24 months when stored at refrigerator temperatures in between administration.

### EXAMPLE 2

### Sucraid® Maintained at 2-8° C.

Sucraid® drug product was prepared by assaying sacrosidase drug substance, and making necessary potency adjustments by adding 50% glycerol/water, followed by making necessary pH adjustments by adding 1 N citric acid. The resultant solution was aseptically filled via 0.2 micron filtration into 118 mL bottles. Bottles were either blow-fill-sealed or pre-sterilized. The pre-sterilized bottles had dropper tips and closures aseptically applied post-filling.

Stability samples were stored at 4° C. or 2-8° C. (nominal 2-8° C.) in the primary drug product containers, LDPE blow fill seal or gamma sterilized LDPE with LDPE dropper tips. Samples were stored on their sides. Lots were typically tested for 0-24 months, with some lots tested to 36, 39, or 48 months. Testing consisted of an enzymatic polarimetry assay that monitors the course of the Sucrose to Glucose+Fructose transformation using the sodium D line at 589 nm.

Based on statistical analysis, a data model 4, different slopes and different intercepts, was indicated, with a highly variable shelf life, determined when the lower confidence interval was exceeded, ranging from 17 to 88 months, for solutions containing about 8313-9650 IU/mL. The average slope was centered at −20.6 IU/mL/month. This loss of potency limits the shelf life of the drug product to 24 months and is necessary to accommodate the drug substance storage needs of the same formulation.

### EXAMPLE 3

### Freezing Point Determination

The freezing point curves of glycerol and water compositions are known, but it was necessary to determine the freezing point of the buffered protein water/glycerol formulation to aid in storage planning. Hence the freezing point of the Sucraid®/sacrosidase formulation was determined using USP <651>, Congealing Temperature. In USP<651>, a liquid sample near its freezing point is immersed in a much cooler liquid. Temperature readings are taken at predefined intervals. The cooling progress is charted. The congealing temperature is accompanied by a plateau in the temperature curve due to enthalpy of freezing energy released at the congealing temperature. USP<651>was modified by using a thermocouple instead of a thermometer to extend the range of the test beyond −20° C. The freezing point was found to be −27° C. to −28° C.

### EXAMPLE 4

### Freeze-Thaw Study

The process of repeated freezing and thawing of a water-based protein solution can destroy a protein's activity due to ice crystal formation and the sheer forces involved. A freeze-thaw study of Sucraid® included 4 complete cycles between thawed 4° C. and completely frozen −80° C., with each freeze being held for 3 days and each thaw being held for 3 days. The results are summarized on Table 1.

TABLE 1

| Elapsed | | |
|---|---|---|
| Time | | Assay Value/ |
| (days) | Event | Description |
| outset | start −80° C. storage | 9533 I.U./ml. [no |
| | | description] |
| 3 | start 4° C. storage (thaw | white, frozen solid |
| | 1) | |
| 6 | aliquot 10 ml. & analyze; | 9533 I.U./ml.; pale- |
| | start −80° C. storage | yellow, clear, viscous |
| | | liquid |
| 9 | start 4° C. storage (thaw | white, frozen solid |
| | 2) | |
| 12 | aliquot 10 ml. & analyze; | 9533 I.U./ml.; pale- |
| | start −80° C. storage | yellow, clear, viscous |
| | | liquid |
| 15 | start 4° C. storage (thaw | [no description] |
| | 3) | |
| 19 | aliquot 10 ml. & analyze; | 9433 I.U./ml.; pale- |
| | start −80° C. storage | yellow, clear, viscous |
| | | liquid |
| 21 | start 4° C. storage (thaw | white, frozen solid |
| | 4) | |
| 26 | aliquot 10 ml. & analyze | 9133 I.U./ml.; pale- |
| | Sucraid for final time | yellow, clear, viscous |
| | | liquid |

As shown in Table 1, the Sucraid®/sacrosidase formulation remained within specification during the first three freeze thaw cycles, indicating a degree of formulation robustness. During the fourth freeze/thaw cycle, the sheer forces associated with aqueous solutions upon freezing and thawing caused a 4.2% loss of its activity during the cycling, confirming that any stored solution must avoid freeze-thaw cycling. Ice crystal formation in aqueous solutions is known to be destructive to protein molecules. (See, J. Pharm. Sci., 88:1325-1330, 1996; see also J. Pharm. Pharmacol., 49:472-477, 1997.)

### EXAMPLE 5

### Sacrosidase Maintained at −20° C. at 5 and 8 Years

A first sacrosidase reference standard was prepared from the Sucraid® drug product by filtering it through a 0.45 μm Corning filter and dispensing 1 mL aliquots into 2 mL glass vials, stoppering, and crimp sealing.

A second reference standard was prepared from the sacrosidase drug product via diafiltration, concentration, Sepharose column purification, and dilution with 50% glycerol/water at pH 4.6. Individual vials were prepared by aliquoting 1.1 mL into 2 mL clear Type I borosilicate glass with 13 mm Teflon faced, gray butyl stoppers and applying an aluminum crimp seal. All reference standards were stored at −10° C. to −25° C. (−20° C. nominal). The initial activities ranged from about 8300-9000 IU/mL.

In the case of the first standard, samples were tested for activity at 0, 48, 72, and 96 months. In the case of the second standard, samples were tested for activity at 0, 12, 24, 36, 48, 60, and 72 months. Testing consisted of an enzymatic polarimetry assay that monitors the course of the Sucrose to Glucose+Fructose transformation using the sodium D line at 589 nm. SLIMSTAT projections indicate common slopes, but different intercepts for each of the two reference standard lots, as shown on FIG. 1.

Based on statistical analysis, a data model 2, common slope but different intercept resulted in a common slope of 1.02 IU/mL/month. This indicates no loss of potency and predicted shelf lives of 537 to 713 months when the upper confidence interval exceeded the upper specification of 9800 IU/mL.

It took periodic evaluation of two separate lots of reference standard over 13 years for the serendipitous discovery that practically infinite stability of enzymatic activity could be achieved if Sucraid® formulation was stored at −20° C. In light of the logistics issues that this discovery addresses, along with the freezing point and freeze-thaw information obtained, sacrosidase/Sucraid® can be stored at −20° C. for at least about seven years with no loss of activity.

The minor increase in slope is due to inherent variability of the drug substance test method. As additional data is collected for the drug substance stability at −20° C., the slope is expected to approach 0 (no loss of activity), consistent with the reference standard experience, and the confidence bands to tighten, leading to even longer predicted shelf lives.

The reference standard drug product and drug substance have equivalent profiles for loss of activity at −20° C., consistent with the fact that they have equivalent glycerol/water formulations and dilute protein concentrations, and also consistent with the independence of container composition (boro silicate glass vs. HDPE) with respect to the sacrosidase protein stability/degradation mechanisms.

An Arrhenius Projection was performed to evaluate the predicted stability of the Sucraid®/sacrosidase shared formulation at −20° C. All available 40° C. and 25° C. data was used. The seven longest running drug product studies at 2-8° C. (4° C. for purposes of Arrhenius projection), all ≧36 months were used. The −20° C. reference standard studies were used, as well as the −20° C. drug substance lots (after normalizing to 8500 IU/mL).

The projection predicts a shelf life of 5970 months based on 95% CI for any sacrosidase/Sucraid® formulation stored at −20° C., consistent with an expectation of near infinite stability when the storage condition of −20° C. is used.

All publications, patents, patent references and references to standard test methods are incorporated by reference herein as though fully set forth.

1. A liquid solution of sacrosidase in an aqueous medium comprising about 35-55% glycerol and the balance, water, wherein the sacrosidase enzymatic activity of the solution is about 7500-19,500 IU/mL, wherein the temperature of the solution is about −18° C. to about −22° C., wherein the enzymatic activity remains constant for at least about 72 months and wherein the sacrosidase is a 513 amino acid polypeptide and is glycosylated.

2. The solution of claim 1, wherein the enzymatic activity is about 8000-9000 IU/mL.

3. The solution of claim 1, wherein the pH is about 4.5-5.0.

4. The solution of claim 1, which does riot contain a preservative.

5. The solution of claim 2, wherein the enzymatic activity remains constant for at least about 96 months.

6-7. (canceled)

8. A container containing closure means wherein the container enclosed the solution of claim 1 that has been introduced into the container under aseptic conditions.

9. A system comprising a container comprising closure means, said container enclosing the solution of claim 1 and a refrigeration means that maintains the solution at about −18° C. to about −22° C.

10. The system of claim 9, wherein the solution is maintained at about −20° C.

11. The solution of claim 1, wherein the weight-weight ratio of glycerol:water is about 1:1.

12. A liquid solution of sacrosidase, having a constant sacrosidase enzymatic activity, in an aqueous medium prepared by a process comprising:
(a) providing a solution having about 7500-19,500 IU/ml of sacrosidase enzymatic activity in an aqueous medium comprising about 35-55% glycerol and the balance water;
(b) lowering the temperature of the solution to about −18° C. to about −22° C.; and
(c) maintaining the solution at about −18° C. to about −22° C. so that the enzymatic activity remains constant, wherein the sacrosidase is a 513 amino acid polypeptide and is glycosylated.

13. The liquid solution of claim 12 wherein the enzymatic activity is about 8000-9000 IU/ml.

14. The liquid solution of claim 12 wherein the pH of the solution is about 4.5-5.0.

15. The liquid solution of claim 13 which does not contain a preservative.

16. The solution of claim 12 wherein the enzymatic activity remains constant for at least about 72 months.

17. An improved liquid solution of sacrosidase having about 7500-19,500 IU/ml of sacrosidase enzymatic activity in an aqueous medium comprising about 35-55% glycerol and the balance water, the improvement comprising:
(a) lowering the temperature of the solution to about −18° C. to about −22° C.; and
(b) maintaining the solution at about −18° C. to about −22° C. so that the enzymatic activity remains constant, wherein the sacrosidase is a 513 amino acid polypeptide and is glycosylated.

18. The liquid solution of claim 17 wherein the enzymatic activity is about 8000-9000 IU/ml.

19. The liquid solution of claim 17 wherein the pH of the solution is about 4.5-5.0.

20. The liquid solution of claim 17 which does not contain a preservative.

21. The solution of claim 17 wherein the enzymatic activity remains constant for at least about 72 months.

22. Sacrosidase preserved in solution in an aqueous medium at about −18° C. to about −22° C., wherein the medium comprises about 35-55% glycerol and the balance, water, having about 7,500-19,500 IU/ml of sacrosidase enzymatic activity, wherein the sacrosidase is a 513 amino acid polypeptide and is glycosylated.
